(19) United States
(12) Patent Application Publication
(10) Pub. No.: US 2019/0142464 A1
(43) Pub. Date: May 16, 2019

(54) TROCAR SEALING SYSTEM CAPABLE OF INTEGRAL INVERSION

(71) Applicant: 5RMED TECHNOLOGY(CHENGDU) CO., LTD., Chengdu (CN)

(72) Inventor: Moshu ZHU, CHENGDU (CN)

(73) Assignee: 5RMED TECHNOLOGY(CHENGDU) CO., LTD.

(21) Appl. No.: 16/243,687

(22) Filed: Jan. 9, 2019

Related U.S. Application Data
(63) Continuation of application No. PCT/CN2017/093606, filed on Jul. 20, 2017.

(30) Foreign Application Priority Data
Aug. 2, 2016 (CN) 201610625809.2

(51) Int. Cl. A61B 17/34

(52) U.S. Cl. CPC A61B 17/3462; A61B 2017/00845

(57) ABSTRACT

The invention discloses a seal membrane with a trocar sealing System capable of integral inversion. Said seal membrane comprises a proximal opening, a distal aperture, and a sealing wall which extends from the distal aperture to the proximal opening, said the sealing wall comprising a proximal surface and a distal surface, said distal aperture formed by a sealing lip for accommodating the inserted instrument and forming a gas-tight seal; said sealing wall, in the lip-adjacent area, is a seamless sealing body with a plurality of normal concave-channel and a plurality of reverse concave-channel surrounding the sealing lip in an alternating manner; said said normal concave-channel is recessed from the proximal surface of the sealing wall toward the distal surface and the opening oriented to the proximal surface.

### CROSS-REFERENCE TO RELATED APPLICATIONS

This application is a continuation of International Patent Application No. PCT/CN2017/093606 with a filing date of Jul. 20, 2017, designating the United States, now pending, and further claims priority to Chinese Patent Application No. 201610625809.2 with a filing date of Aug. 2, 2016. The content of the aforementioned applications, including any intervening amendments thereto, are incorporated herein by reference.

### TECHNICAL FIELD

The present invention relates to a minimally invasive surgical instrument, and in particular, to a trocar sealing element.

### BACKGROUND OF THE PRESENT INVENTION

A trocar is a surgical instrument, that is used to establish an artificial access in minimally invasive surgery (especially in rigid endoscopy). Trocars comprise in general a cannula and an obturator. The surgical use of trocars generally known as: first make the initial skin incision at the trocar insertion site, then insert the obturator into the cannula, and then together they facilitated penetration of the abdominal wall through incision into the body cavity. Once penetrated into the body cavity, the obturator is removed, and the cannula will be left as access for the instrument get in/out of the body cavity.

In rigid endoscopy surgery, it is usually necessary to establish and maintain a stable pneumoperitoneum for the sufficient surgical operation space. The cannula comprises a sleeve, an outer body, a seal membrane (also known as instrument seal) and a duck bill (also known as closure valve). Said cannula providing a channel for the instrumentation in/out of the, body cavity, said outer body connecting the sleeve, the duck bill and the seal membrane into a sealing system; said, duck bill normally not providing sealing for the inserted instrument, but automatically closing and forming a seal when the instrument is removed; said seal membrane accomplishing a gas-tight seal against the instrument when it is inserted.

In a typical endoscopic procedure, it is usually set up 4 trocars (access), i.e. 2 sets of small diameter cannula (normally 5 mm in diameter), and 2 sets of large diameter cannula (normally 10-12 mm in diameter). Instruments, in general passing through a small cannula are only for ancillary works; herein one large cannula as an endoscope channel, and the other large cannula as the main channel for surgeon to perform surgical procedures. Through said main channel thereof, 5 mm diameter instruments used in approximately 80% of the procedure, and said large cannula used in approximately 20% of the procedure; furthermore, 5 mm instruments and large diameter instruments need to be switched frequently. The small instruments are mostly used, so that the sealing reliability of which is more important. The large instruments are more preferably used in a critical stage of surgery (Such as vascular closure and tissue suturing), therein switching convenience and operational comfort are more important.

FIG. 1 and FIG. 2 depict a typical 12 mm diameter cannula 700. Said cannula 700 comprises a lower housing 710, an upper housing 720, a seal membrane 730 which sandwiched between the lower housing 710 and the upper housing 720, and a duckbill seal 750. Said lower housing 710 including center hole 713 defined by an elongated tube 711. Said upper housing 720 including the proximal hole 723 defined by the inner wall 721 Said membrane 730 including a proximal opening 732, a distal aperture 733, a sealing lip 734, a frustum sealing wall 735, a flange 736 and an outer floating portion 737. Said distal opening 733 formed by a sealing lip 734. Said sealing lip 734 defining a longitudinal axis 741, the transverse plane 742 substantially perpendicular to said axis 741; define the angle between the rotary-generating line (or generatrix) of the frustum sealing wall 735 and the transverse plane 742 as a guide angle ANG1.

As illustrated in FIG. 1, when a 5 mm diameter instrument inserted, it is approximately considered that only hoop force generated by the deformation of the sealing lip 734, ensures a reliable seal for the instrument. It is nevertheless favorable to operate the instrument from various extreme angles in surgery. There's a lot space left for the 5 mm-instrument to move radially in the 12 mm diameter cannula, so that greater radial force would be taken by the sealing lip 734. Therefore, the sealing lip 734 should have sufficient hoop force for the inserted 5 mm diameter instrument to ensure its sealing reliability thereof.

As illustrated in FIG. 2, drawing a cylinder of Di (Di>5 mm) to cut the sealing wall 735 forms an intersecting line 738. It is easy to understand for those skilled in the art, when an Di diameter instrument is inserted, the strain (stress) of said sealing wall 735 in the area from the sealing lip 734 to the intersecting line 738 will be larger, so the area refer to as lip-adjacent area(or concentration stress area). While the strain (stress) of said sealing wall 735 from the intersecting, line 738 to the flange 736 is small. However, the different diameter (Di value) makes the boundary range of the lip-adjacent area (or concentration stress area) change larger or smaller. For the convenience of quantification, it is defined when Di is designed as the maximum diameter of the surgical instrument passing through the seal membrane, the area from the sealing lip 734 to the intersection line 738 is the lip-adjacent area.

As illustrated in FIG. 3, when a large diameter instrument is inserted (e.g. 12.8 mm), the sealing lip 734 will expand to a suitable size to accommodate the inserted instrument; said sealing wall 735 is divided into two portions: a conical wall 735c and a cylindrical wall 735d; said cylindrical wall 735d wrapped around the outer surface of the instrument to form a wrapped area with a high concentration of stress. Defining the intersecting line of the conical wall 735c and the cylindrical wall 735d as intersecting line 738a. When the instrument, is removed, said sealing wall 735 return to natural state, and said intersecting line 738a spring-back to a ring radius of Dx, defined as intersecting line 738b, (not shown in FIG.); said intersecting line 738b is a bending boundary line when inserting a large diameter instrument. The angle between the rotary generating line of said conical wall 735c and the transverse plane 742 defines as ANG2, ANG2>ANG1; that is, when the large-diameter instrument is inserted, said sealing wall 735 rotates and stretch around its intersection line of said flange 736. Defining the height of the cylindrical wall 735d as Ha, not a fixed value; the factors such, as different size of said distal aperture, different size of said sealing lip, different thickness of said sealing wall, different said guide angle or different diameter of inserted instrument, make Ha different.

The instrument inserted into the sealing membrane and moved during surgical procedure, there is large frictional resistance between the wrapped area and the inserted instrument. Said large frictional resistance is normally easy to cause the, seal inversion, poor comfort of performance, fatigue performance, even result in cannula insecurely fixed on the patient's abdominal wall etc., such that the performance of cannula assembly is affected.

Among the defects caused by the large frictional resistance, the seal inversion is one of the most serious problems that affecting the performance of the cannula. As illustrated in FIG. 4, when a large diameter instrument is removed, easily cause seal inversion. When inversion happened, said sealing wall 735 divided into a cylindrical wall 735e, a conical wall 735f, and a conical wall 735g; said cylindrical wall 735e wrapped around the outer surface of the instrument to form a wrapped area with a high concentration of stress. Defining the height of the cylindrical wall 735e to be Hb, normally Hb>Ha; that is, the frictional resistance when the instrument is removed greater than it when the instrument is inserted, this difference affects the surgeon's operating feeling and even make the surgeon confused. More seriously, the inversion of the seal membrane may stretch into the proximal hole 723, that is the seal membrane positioned between the instrument and the inner wail 721 gets completely jammed. Measures for preventing the seal inversion are respectively disclosed in U.S. Pat. No. 7,112,185 and U.S. Pat. No. 7,591,802, and those measures can effectively reduce the probability of inversion but not completely solve the problem.

The simplest way to reduce the frictional resistance is reducing the coefficient of friction between the two contacting surfaces with grease, but the reliability of this way is not good. During procedures, due to instruments long-term repeated scraping with the seal membrane and repeated switching, it is easy to erase the grease off and carried away, resulting in bad lubrication.

A protector assembly adjoined by a seal membrane is disclosed in U.S. Pat. No. 5,342,315. Said protector to permit the sharp edge of the instrument to pass through the opening in the seal membrane without causing damage to the seal membrane, and the surface friction coefficient of the protector assembly is smaller than the surface friction coefficient of the seal membrane, which results in less frictional drag, but the lip-adjacent area is normally not completely covered by the protector assembly.

A seal member with ribs (or projections) is disclosed in U.S. Pat. No. 5,827,228, that is a plurality of spaced ribs provided to extend outwardly from center hole to reduce surface contact between the inserted instrument and the seal member, and thereby reducing the frictional resistance, a similar ribs which disclosed in EP0994740 also reducing surface contact and strengthen the tensile of the seal member oriented to axial.

A sealing element comprising a flexible wall closed annularly with the edges foldable in a wave-like manner is disclosed in U.S. Pat. No. 7,842,014, wherein the wall bears a wave-like sealing lip and is a wavy pleated seal body, in such manner it can enlarge hoop circumference, and reduce the hoop force to a certain extent.

Chinese invention application CN101480354A (currently rejected) discloses a seal member containing an easily deformable groove, wherein is characterized in that it has a plurality of easily deformable grooves on the conical surface of the seal member from the sealing lip; said the thickness of the deformable groove wall is much smaller than the thickness of the conical surface wall, primary take advantage of the elongation of the deformable groove to accommodate the inserted large diameter instrument.

Although, in the prior art many solutions for reducing the frictional resistance have been disclosed, these solutions basically only propose measures from one certain factor affecting frictional resistance, the effect of which is small or not obvious. Some modifications solved a certain defects may lead to cause another bug. Such as, reinforcing ribs on the seal membrane to reduce surface contact, meanwhile strengthen the tensile of the seal membrane; or a deformable groove with a thickness much smaller than that of a truncated conical surface can cause the deformable groove to be easily damaged; due to the adoption of said wave-like sealing lip which enlarge hoop circumference, the sealing reliability will be sacrificed when a 5a mm diameter instrument is inserted, if the wave-like sealing lip is used but without enlarge hoop circumference, the wave-like sealing lip will lose its improvement effect. In summary there are many factors affecting the frictional resistance, and the comprehensive effects of various factors must be considered in the perspective of mechanics and tribology.

The seal membrane is preferably produced from rubber such as natural rubber, silicone or polyisoprene, its mechanical properties including super elastic and viscoelastic. Although the mechanical model of the rubber deformation process is complicated, it can still apply the generalized Hooke's law to describe approximatively its elastic behavior; and Newton's internal friction law to describe the viscous behavior. Research suggests that the main factors affecting the friction of the two surfaces in contact between the rubber and the instrument include: the smaller the friction coefficient of said two surfaces, the smaller the friction is; the better lubrication condition of said two surfaces in contact, the friction smaller is; the smaller normal pressure of said two surfaces, the friction smaller is. Comprehensively considering the above factors, the present invention proposes better solutions for reducing the frictional resistance between the seal membrane and the inserted instrument.

In addition to said frictional resistance greatly affecting the performance of the cannula assembly, the stick-slip of the seal membrane is another main factor affecting the performance of trocar. Said stick-slip means that when the instrument moves longitudinally in the sleeve, the sealing lip and lip-adjacent area sometimes are relatively statically attached to the instrument (at this point, the friction between the instrument and the seal membrane is mainly static friction.); but sometimes it produced a relatively slippery phenomenon with the instrument (at this point, the friction between the instrument and the seal membrane is mainly dynamic friction.); and said static friction is much greater than said dynamic friction. The two frictions alternately occur, which causes the movement resistance and speed of the instrument in the seal membrane to be unstable. It is easy to be understood for those skilled in the art, that in minimally invasive surgery the surgeon can only use surgical instruments to touch (feel) the patient's organs and observe a part of the working head of the instruments through endoscopic image system. In this case where the vision is limited and it cannot be touched, the surgeon typically uses the feedback of the resistance when moving instruments as one of the information to judge whether the operation is abnormal nor not. The stick-slip affects the comfort of operation, the accuracy of positioning, and even induces the surgeon to make false judgment.

During the surgical application of the cannula, the stick-slip is difficult to avoid, but can be, reduced. Researches have shown that said stick-slip is affected by two main factors: one is that the smaller the difference between the maximum static friction and the dynamic friction, the weaker the stick-slip is; the other is that the larger the axial tensile stiffness of the seal membrane, the weaker the stick-slip is. Avoiding excessive the hoop force between the seal membrane and the instrument, reducing the two surfaces contacted, maintaining good lubrication, respectively, can reduce the difference between the maximum static friction and the dynamic friction, thereby reducing stick-slip, meanwhile, increasing the axial tensile stiffness of the seal membrane also helps to reduce the stick-slip phenomenon. The invention also proposes measures for improving stick-slip.

In summary, so far, there is no cannula that can effectively solve the said problems.

### SUMMARY OF PRESENT INVENTION

One object of the invention is to solve the problem of the seal inversion from another perspective: take measures to reduce the probability of the seal inversion, at the same time, completely solve the problem of complete jam, and take measures to reduce frictional resistance and improve comfort of performance after inversion.

To design an integral invertible trocar seal assembly need to solve two major hazards caused by the inversion of the seal membrane described in the background of the invention. One of the main hazards caused by the inversion of the seal membrane is that the elastic material accumulates between the instrument and the floating ring (the center hole of the outer body) abject to the seal membrane, resulting in blockage, the occurrence of which hazard is mainly due to the lack of reserved clearance for inversion In one aspect of the present invention, designing a seal system for trocar: includes an upper body, a lower body, a center hole axially aligned with said upper body and said lower body, also includes a seal membrane assembly mounted between said upper body and said lower body. Said seal membrane assembly comprises a seal membrane, a protection device, the first retainer ring and the second retainer ring. Said seal membrane comprises a distal aperture, an inner seal body, a flange, an outer floating portion and a proximal end. Said protection device comprises a flange and a protector said seal membrane and said protect device are sandwiched between the first retainer ring and the second retainer ring, and one side of the flange of said protection device is in close contact with the flange of said seal membrane, while the other side in close contact with the first retainer ring. The geometric size of said first retainer ring conforms to the following equation:

Rᵢ≥Rᵢₙ+Tₛ+Tₚ+δ

H≥Hₛ+ΔL
Ri=radius of center hole of the first retainer ring
Rin=the largest radius designed for the surgical instrument passed through the seal membrane
Ts=thickness of sealing wall
Tp=thickness of protector
δ=reserved clearance
H=height of the first retainer ring
Hs=vertical height of a frustum section of the seal membrane
ΔL=elongation of a frustum section of the seal membrane when working

Another main hazard caused by the, inversion of the seal membrane is that it causes iction to abnormally increase and unstable the instrument when moving out from the patient's body, and affect the comfort of use. There are two main factors: first, the function of the protector after the inversion is completely lost, the wrapped area on the back of the instrument and the seal membrane increases, which leads to frictional resistance increased; second, the large frictional resistance after inversion is prone to “stick-slip” phenomenon.

Therefore, it is necessary to design a trocar seal membrane, which comprises a proximal opening, a distal aperture, and a sealing wall from the distal aperture extending, to the proximal opening, said distal aperture formed by a sealing lip for accommodating the inserted instrument arid forming a gas-tight seal. Said the sealing wall includes a proximal surface and a distal surface. Said seal membrane can ensure a reliable seal for the inserted 5 mm instrument, and reduce frictional resistance and improve stick-slip when a large-diameter instrument is inserted.

As described in the background, the wrapped area formed by the sealing lip and the lip-adjacent area when a large diameter instrument inserted, is the major factor cause of frictional resistance. For reducing said frictional resistance, comprehensive consideration should be given such as reducing the radial stress between the instrument and the seal membrane, reducing said wrapped area, and reducing the actual contact area of the two surfaces. It is easy to understand for those skilled, in the art that in accordance with the generalized Hooke's law and Poisson effect, enlarge hoop circumference, and reduce hoop strain (stress), thereby reducing radial strain (stress). But it should be noted that it is impossible to enlarging the hoop circumference in order to reduce the strain of the sealing lip which will result in reduced sealing reliability when applying 5 mm instruments. Since the stress in the lip-adjacent area is highly concentrated when applying a large diameter instrument, the hoop circumference of the lip-adjacent area should be rapidly increased. In regard to outside the lip-adjacent area, since the, strain (stress) is small, it is not necessary to adopt measures to enlarge the hoop circumference. In, addition, enlarging the hoop circumference, in the meantime increasing the axial tensile stiffness in the lip-adjacent area and maintain good lubrication (reducing difference between the maximum static friction and dynamic friction), thereby the stick-slip in the lip-adjacent area is improved.

In one aspect of the present invention, said seal membrane comprises a proximal opening, a distal aperture, and a sealing wall from the distal aperture extending to the proximal opening, said sealing wall with a proximal surface and a distal surface, said distal aperture formed by a sealing lip for accommodating the inserted instrument forms a gas-tight seal, said sealing wall in the lip-adjacent area, is a seamless sealing body with a plurality of normal concave-channels and a plurality of reverse concave-channels surrounding the sealing lip in an alternating manner. Said normal concave-channels are recessed from the distal surface of the sealing wall toward the proximal surface and the opening oriented to the distal surface. The shape of the normal concave-channels from the perspective of distal surface is represented as a hollow convex-rib that is raised from the distal surface. Said normal concave-channels extend laterally outward from the sealing lip, and the depth of said concave-channels gradually increases in the lip-adjacent area; while the depth of said concave-channels gradually decreases outside the lip-adjacent area. In the lip-adjacent area, said seal membrane includes 8 normal concave-channels with the approximately U-shaped section and 8 reverse concave-channels with the approximately U-shaped section. Said seal membrane includes a flange at which the sealing wall extendedly intersects, or simultaneously the sealing wall and said concave-channels extendedly intersect, and an outer floating portion including at least one lateral pleat extending from the flange to the proximal opening.

Said the seal membrane with concave-channels has the functions of enlarging hoop circumference, reducing the wrapped area, reducing the actual contact area of the two surfaces between the instrument and the seal membrane, improving lubrication reliability, increasing the axial tensile stiffness, etc., thereby, the frictional resistance and the stick-slip can be greatly reduced, and the probability of inversion is reduced and the comfort of application is improved. While said concave-channels, the depth of which gradually increases in the lip-adjacent area; while the depth of which gradually decreases, outside the lip-adjacent area, which can simplify mould design, improve the efficiency of the seal membrane processing; reduce the space occupied by the lateral movement of the seal membrane assembly, so that the size of trocar can be designed to be smaller; reduce the material accumulation between the seal membrane and the instrument after inversion and the actual contact area of the two surfaces.

In another aspect of the present invention, said seal membrane comprises a proximal opening, a distal aperture, and a sealing wall from the distal aperture extending to the proximal opening, said sealing wall with a proximal surface and a distal surface, said sealing wall in the lip-adjacent area, is a seamless sealing body with a plurality of normal concave-channel and a plurality of reverse concave-channel surrounding the sealing lip in an alternating manner. Said normal concave-channel is recessed from the proximal surface of the sealing wall toward the distal surface and the opening oriented to the proximal surface. The shape of the normal concave-channel from the perspective of distal surface is represented as a hollow convex-rib that is raised from the distal surface. Said normal concave-channel includes two side sealing-walls and an outer sealing-wall defined by the two side sealing-walls, said outer sealing-wall has a proximal surface and a distal surface, and said outer sealing-wall includes solid-rib rising to the distal surface. In an alternative embodiment, said normal concave-channel has U-shaped solid-rib rising from the bottom of the concave-channel toward the distal surface. In another alternative embodiment, said normal concave-channel has rectangular solid-rib rising from the bottom of the concave-channel toward the distal surface. The main function of said U-shaped solid-rib or rectangular solid-rib is: when inversion happened, the raised solid-rib reduces surface direct contact between the instrument and the inverted seal membrane, thereby reducing the frictional resistance. At the same time, the recessed area between the solid-ribs has a certain role in storing grease. When the seal membrane is turned over, the friction between the instrument and the seal membrane, first take away the grease on the raised solid-ribs, while the grease in the recessed area between the solid-ribs can be added to the surface of the solid-rib as the instrument moves, thereby improve lubrication reliability in a certain degree after inversion. However, arbitrary solid-rib cannot extend to the sealing lip, it should be kept away from the sealing lip as, much as possible to prevent from increasing the hoop force in adjacent areas.

It is believed that the above invention or other objects, features and advantages, will be understood with the drawings and detailed description.

### DESCRIPTION OF THE DRAWINGS

A more complete appreciation of this invention and many of the attendant advantages thereof will be readily apparent as the same becomes better understood by reference to the following detailed description, where:

FIG. 1: shows a simulated distorted view of the cannula with the 5 mm diameter instrument inserted in the prior art;

FIG. 2: shows a detailed view of the seal membrane 730 in the prior art;

FIG. 3: shows a simulated distorted view of the cannula with the 12.8 mm diameter instrument inserted in the prior art;

FIG. 4: shows a simulated distorted view of the cannula with the 12.8 mm diameter instrument removed in the prior art;

FIG. 5: shows a 3D perspective partial sectional view of the cannula in the invention;

FIG. 6: shows an exploded view of the seal membrane assembly of the cannula in FIG. 5;

FIG. 7: shows a 3D perspective partial sectional view of the seal membrane assembly in FIG. 6;

FIG. 8: shows a 3D perspective view of the seal membrane without the proximal end and floating portion in FIG. 6;

FIG. 9: shows a sectional view along line 9-9 in FIG. 8;

FIG. 10: shows a reversed 3D perspective sectional view of the seal membrane in FIG. 8;

FIG. 11-12: shows a segmentation view of the seal membrane after the circumferential cutting separation in FIG. 8;

FIG. 13: shows a simulated distorted view of the seal membrane assembly with the 12.8 mm instrument inserted in FIG. 7;

FIG. 14: shows a reverse 3D perspective view of the seal membrane assembly in FIG. 13;

FIG. 15: shows a sectional view along-line 15-15 in FIG. 13

FIG. 16: shows a simulated distorted view of the seal membrane assembly with the 12.8 mm instrument removed in FIG. 7;

FIG. 17: shows a partial view along-line 17-17 in FIG. 16

FIG. 18: shows a partial view along-line 18-18 in FIG. 16

FIG. 19: shows a 3D perspective view of the seal membrane of another embodiment according to the invention;

FIG. 20: shows a 3D perspective view of the seal membrane of another embodiment according to the invention; and

FIG. 21: shows a partial view along-line 21-21 in FIG. 19

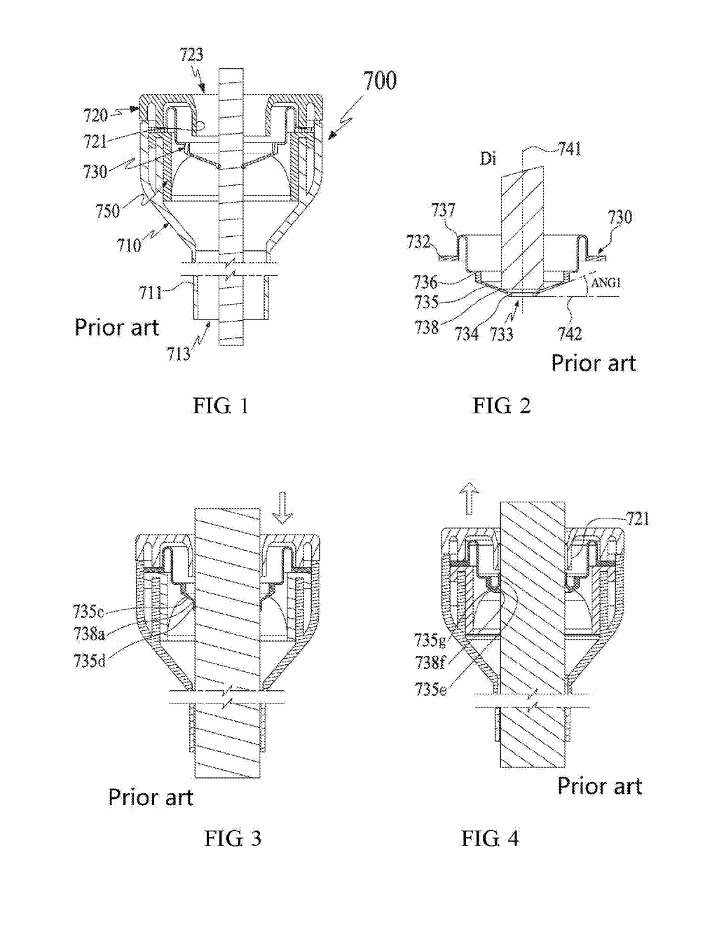

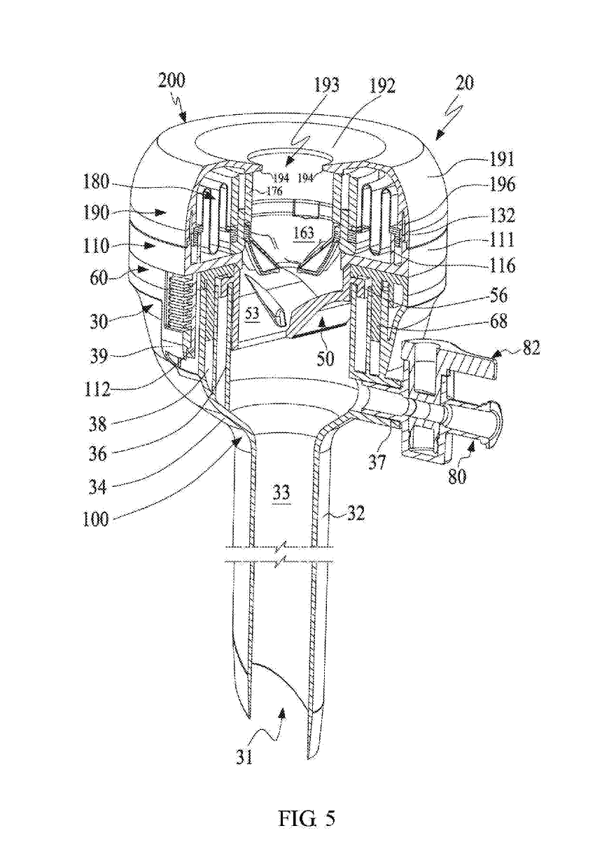

### DETAILED DESCRIPTION OF PREFERRED EMBODIMENTS

Embodiments of the invention are disclosed herein, however, it should be understood that the disclosed embodiments are merely examples of the invention, which may be implemented in different ways. Therefore, the invention is not intended to be limited to the detail shown, rather, it is only considered as the basis of the claims and the basis for teaching those skilled in the art how to use the invention.

FIG. 2 shows an, overall view of the structure of trocar. A typical trocar comprises an obturator 10 (not shown) and a cannula 20. The cannula 20 comprises an open proximal end 192 and an open distal end 31. In a typical embodiment, said obturator 10 passes through said cannula 20, together they facilitated penetration of the abdominal wall through incision into the body cavity. Once penetrated into the body cavity, the obturator 10 is removed, and the cannula 20 will be left as access for the instrument get in/out of the body cavity. Said proximal end 192 in the external position of the patient and said distal end 31 in the internal position. A preferred cannula 20 can be divided into the first seal assembly 100 and the second seal assembly 200. Locking receptacle 39 in said seal assembly 100 can be locked with snap-in projection 112 in said seal assembly 200. The cooperation of snap-in projection 112 and the locking receptacle 39 can be, quick release by one hand. The main purpose is for convenience of taking out tissues or foreign matter from the patient in the surgery. There are multiple ways to implement the quick release connection of said seal assembly 100 and assembly 200. In addition to the structure, shown in this embodiment, a threaded connection, a rotary snap-in or other quick lock structure also may be applied. Alternatively, said assembly 100 and assembly 200 can be designed as a structure that can not be split quickly.

FIG. 5 shows the composition and assembly relationship of the first seal assembly 100, The lower body 30 includes an elongated tube 32, which defines the sleeve 33 passed through the distal end 31 and is connected to the outer housing 34. Said lower body 30 comprises an inner wall 36 supporting duck bill seal and a valve bore 37 that communicates with the inner wall 36. The plunger 82 mounted in the valve body 80, the said two are mounted into said valve bore 37. The flange 56 of the duck bill seal 50 is sandwiched between the inner wall 36 and the lower cover 60. There are various ways of fixing between the lower cover 60 and the lower body 30, such as the interference fit, ultrasonic welding, glue bonding, and snap fastening. 4 cylinders 68 of said lower cover 60, in this embodiment, 4 holes 38 of said lower body 30 are adopted to interference fit, so that the duckbill seal 50 is in the compressed state. Said tube 32, said the inner wall 36, said duck bill seal 50, said valve body 80 and said plunger 82 together are comprised the first chamber. Said duck bill seal 50, in this embodiment, is a single-slit, while other types of closure valves may also be used, including flapper valves, multi-silted duck bill valves. When the instrument is passed through said duck bill seal 50, the duckbill 53 will be opened, but it generally does not provide a complete seal against the instrument. When the instrument is removed, said duckbill 53 closed and substantially prevents insufflation fluid from escaping through the first chamber.

FIG. 5 shows the composition and assembly relationship of the second seal assembly 200. The seal membrane assembly 180 is, sandwiched between the upper cover 110 and the upper body 190. The proximal end 132 of the seal membrane assembly 180 is secured between the inner ring 116 of the upper cover 110 and the inner ring 196 of the upper body 190. There are various secured ways between the upper cover 190 and the upper body 110, such as the interference fit, ultrasonic welding, glue bonding, and snap fastening. The connection method, shown in this embodiment, is the outer shell 191 of the upper body 190 and the outer shell 111 of the upper cover 110 are secured by ultrasonic welding so that the proximal end 132 of the seal membrane assembly 180 is in the compressed state. The center hole 113 of said upper cover 110, said inner ring 116, and said seal membrane assembly 180 together are comprised the second chamber.

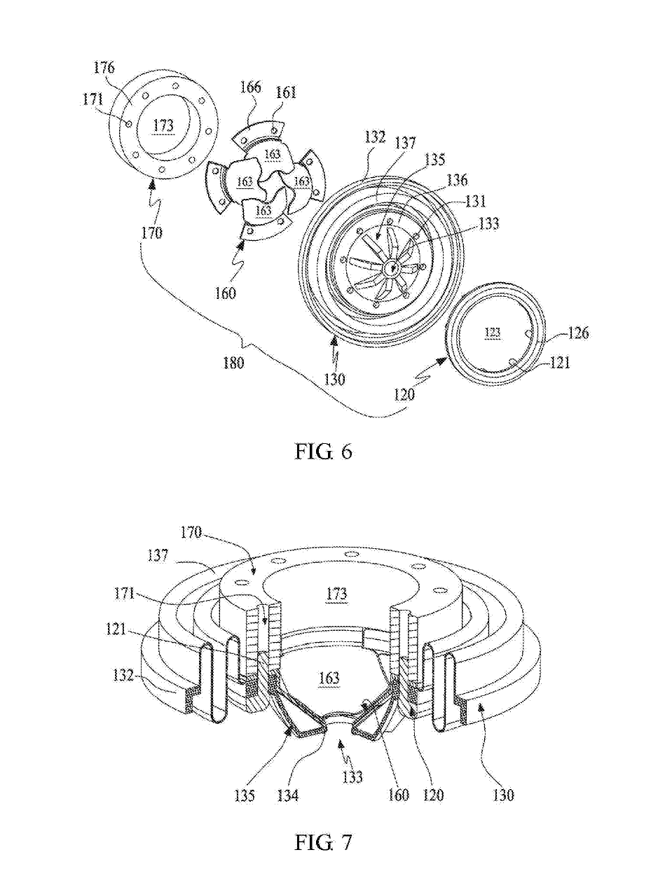

FIG. 6-7 illustrate the composition and assembly relationship of said seal membrane assembly 180, which including a lower retainer ring 120, a seal membrane 130, a protection device 160 and an upper retainer ring 170. Said the seal membrane 130 and said protection device 170 are sandwiched between the lower retainer ring 120 and the upper retainer ring 125, moreover, the cylinder 121 of the said lower retainer ring 120 is aligned, with corresponding holes, on other components in said seal membrane assembly 180. Said cylinder 121 and the bore 171 of the upper retainer ring 170 are adopted to interference fit, so that the whole seal membrane assembly 180 in the compressed state. Said protection device 160 includes 4 protectors 163 arranged so as to protect a center sealing body of said seal membrane 130, herein permit the sharp edge of the instrument to pass through without causing perforations or tears to the seal membrane 130.

Said seal membrane 130 includes a proximal opening 132, a distal end aperture 133, and the sealing wall extending from the distal end to the proximal end, said sealing wall including a proximal surface and a distal surface. Said aperture 133 formed by a sealing lip 134 for accommodating an inserted instrument and forming a gas-tight, seal. Said sealing lip 134, in the present embodiment, is approximately circular, but said sealing lip 134 may be non-circular.

Said the seal membrane 130 also including the flange 136; The sealing wall 135 has one end connected to the sealing lip 134 and the other end connected to the flange 136; the floating portion 137 has one end connected to the flange 136 and the other end connected to said proximal end 132. Said flange 136 for mounting the protector device 160. Said floating portion 137 including one or several plurality of radial (lateral) pleats, so that the entire seal membrane assembly 180 can float in the assembly 200.

Said assembly 180 can be made from a variety of materials with a range of different properties. For instance, said seal membrane 130 is made of, a super elastic material such as silicone or polyisoprene; said protector device 160 is made of a semi-rigid thermoplastic elastomer; and said second retainer ring 120 and said first retainer ring 170 are made of a relatively hard rigid material such as polycarbonate.

An Integral invertible trocar seal assembly need to solve the, two major hazards caused by the inversion of the seal membrane described in the background of the invention. One of the main hazards caused by the inversion of the seal membrane is that the elastic material accumulates between the instrument and the floating ring (the center hole of the outer body) abject to the seal membrane, resulting in blockage, the occurrence of which hazard is, mainly due to the lack of reserved clearance for inversion. In one aspect of the present invention, by increasing the inner diameter of the center hole 173 of the first retainer ring 170, a sufficient inversion space, is reserved to ensure that, the sealing membrane does not become stacked into blocked between the first retainer ring and the instrument after the seal membrane inversion. It will be understood by those skilled in the art, the inner diameter of the proximal hole 193 of the upper body 190 cannot be increased. Since the inner diameter of the proximal hole 193 is increased, it is inevitable that when a 5 mm diameter instrument is inserted, the lateral movement space of the instrument is increased, resulting in a decrease in sealing reliability. Therefore, the inverted seal membrane should be prevented from entering the, proximal hole 193.

One of the simple methods is to increase the height of the first retainer ring 170, and completely limit the inverted seal membrane in the central hole 173 of the first retainer ring. Those skilled in the art readily conceive that the end surface 194 of the proximal hole 193, and the maximum diameter thereof should be larger than the inner diameter of the center hole 173 to prevent the end surface 194 from falling into the center hole 173. Those skilled in the art readily conceive that said end surface 194 can be a complete curved surface or be constructed of a series of discontinuous ribs (in order to prevent injection shrinkage).

Optionally, said first retainer ring conforms to the following equation:

Rᵢ≥Rᵢₙ+Tₛ+Tₚ+δ

H≥Hₛ+ΔL
Ri=radius of center hole of the first retainer ring
Rin=the largest radius designed for the surgical instrument passed through the seal membrane
Ts=thickness of sealing wall
Tp=thickness of protector
δ=reserved clearance
H=height of the first retainer ring
Hs=vertical height of a frustum section of the seal membrane
ΔL=elongation of a frustum section of the seal membrane when working

Normally the thickness of the sealing wall 135 is 0.5˜0.8 mm, the thickness of protector 163 0.1˜0.3 mm, reserved clearance δ is usually twice as thick as the sealing wall 135, e.g. δ≥1 mm. Elongation of a frustum section of the seal membrane when working ΔL is defined by the structure and size of the sealing wall 135 of the seal membrane itself, for example, different thickness of the sealing wall 135, different elongation ΔL.

Another main hazard caused by the inversion of the seal membrane is that it causes friction to abnormally increase and unstable the instrument when moving out from the patient's body, and affect the comfort of use. There are two main factors: first, the function of the protector after the inversion is completely lost, the wrapped area on the back of the instrument and the seal membrane increases, which leads to frictional resistance increased; second, the large frictional resistance after inversion is prone to “stick-slip” phenomenon.

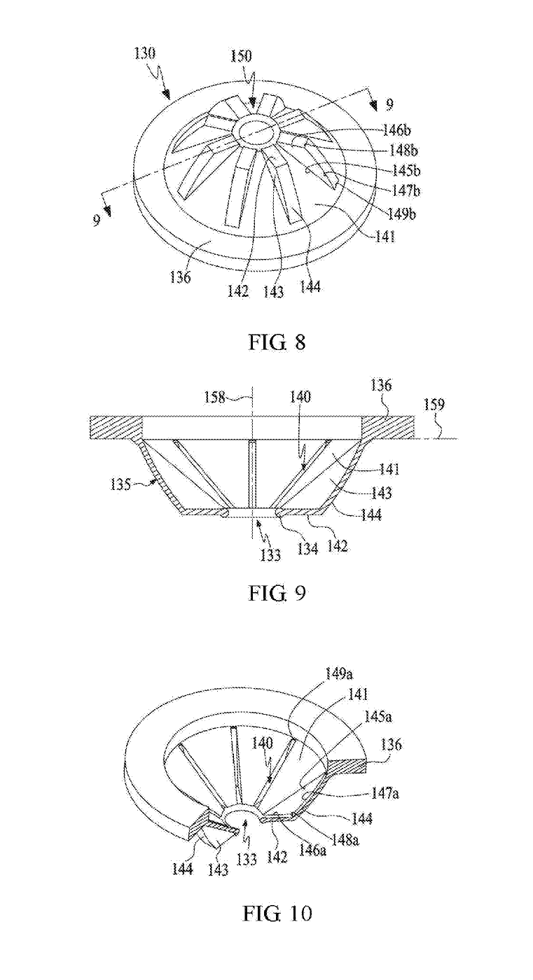

FIG. 8-10 show more detailed depiction of the seal membrane 130 in the first embodiment of the invention. In order to reduce the production cost, the seal membrane 130 is preferably designed as a monolithic part, but can also be designed as an, inner seal body and an outer floating portion, separated from the flange 136. The first embodiment is mainly directed to the improvement of, the inner seal body. To simplify the description, the outer floating portion and the proximal end are not shown in the subsequent description of the seal membrane.

Defining the axis of said sealing lip 134 as the longitudinal axis 158, and a transverse plane 159 that is generally perpendicular to the longitudinal axis 158. Said sealing wall 135, which can be approximately frustum, approximately hemispherical, or an irregularly rotating surface. In, this embodiment said wall 135 is formed in an approximately conical arrangement surrounding the sealing lip 134. Said wall 135 including an inner sealing-wall 141, an outer sealing-wall 142 and a side sealing-wall 143. Said inner sealing-wall 141 extends laterally from the sealing lip 134 to the flange 136; said an outer sealing-wall 142 extends laterally from the sealing lip 134 to tilted-sealing-wall 144; while said tilted-sealing-wall 144 and said inner sealing-wall 141(or the flange 136) are intersected. The first side of said side sealing-wall 143 intersects the inner sealing-wall 141 and form a line 145a, 145b; the second side of said side sealing-wall 143 intersects the outer sealing-wall 142 to form a line 146a, 146b; the third side of said side sealing-wall 143 intersects the tilted-sealing-wall 144 to form a line of intersection 147a, 147b. One end of said tilted-sealing-wall 144 intersects the outer sealing-wall 142 to form a line 148a, 148b; the other end of said tilted-sealing-wall 144 intersects the inner sealing-wall 141 to form a line 149a, 149b.

Referring to FIG. 8-10, in the lip-adjacent area: said 2 adjacent side sealing-wall 143 and the outer sealing-wall 142 there between form a concave-channel that is recessed from the proximal surface toward the distal surface and the opening oriented to the proximal surface, which is defined as the normal concave-channel 140; meanwhile, said 2 adjacent side sealing-wall 143 and the inner sealing-wall 141 there between form a concave-channel that is recessed from the distal surface toward the proximal surface and the opening oriented to the distal surface, which is defined as the reverse concave-channell 50. In the lip-adjacent area, said inner sealing-wall 141, said side sealing-wall 143 and said outer sealing-wall 142 forms a series of normal concave-channel and, reverse concave-channel, and said normal concave-channel and reverse concave-channel alternately distributed in circular array around the sealing lip 134, extending laterally outward and gradually increasing in axial depth. The measurement method of the concave-channel depth is: The shortest distance from the point at the bottom of the concave-channel recess to the main rotary wall along in the direction of longitudinal axis. In the lip-adjacent area, the adjacent normal concave-channel and the reverse concave-channel sharing a mutual side sealing-wall and a series of concave-channels with the normal and reverse alternately distributed with increasing axial depth and form a seamless sealing wall 135.

Defining the angle between said intersection line 145a (145b) and said transverse plane surface 259 as a, which is called the guide angle α. The angle formed by the intersection lines 145a and 146a (or 145b and 146b) is defined as θ. The intersection of the two intersection lines (i.e. the apex of the angle θ) may be on the sealing lip 134; or the virtual extension lines of the two intersection lines intersect the inside of the sealing lip 134.The angle formed by the intersection lines 145a and 147a (or 145b and 147b) is defined as β. In the lip-adjacent area, the side sealing-wall 143 is a surface defined by both sides and extending laterally outward from the sealing lip 134 and gradually widening; while outside the lip-adjacent area, said side sealing-wall 143 is a tapered region defined by the edges, said side sealing-wall 143 is approximately obtuse triangular near the boundary of the lip-adjacent area. That is, when said normal concave-channel 140 extending laterally outward, the depth of it gradually increases in the lip-adjacent area, while gradually decreases outside the lip-adjacent area. Moreover, the 2 adjacent side walls 143 and the inner sealing-wall 141 there between only a distinct reverse concave-channel is formed in the lip-adjacent area, while no distinct reverse concave-channel is formed outside the lip-adjacent area. From the perspective of the distal surface, said normal concave-channel 140 is shaped as a hollow convex-rib that is raised on the distal surface. Said hollow convex-rib extends laterally outward from the sealing lip and gradually increase in height, and abrupt change occurs near the boundary of the lip-adjacent area, and the height thereof rapidly decreases.

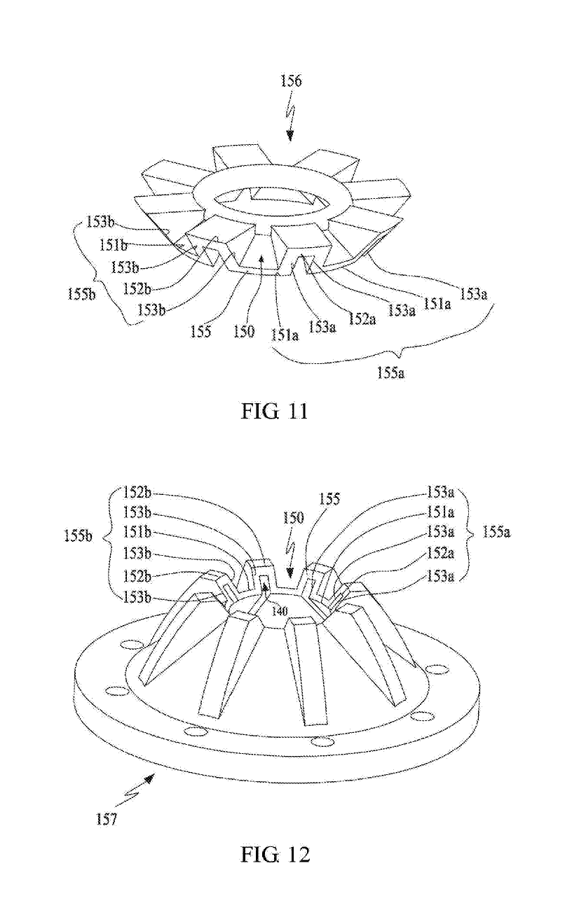

FIG. 11-12 illustrates, in an alternative embodiment, the thickness of normal concave-channel and reverse concave-channel is substantially uniform, that is the thickness of the inner side wall 141, the outer sealing-wall 142 and the side sealing-wall 143 is substantially equal. Said substantially uniform thickness causes the deformation of the sealing wall 135 to be substantially uniform. However, said substantially uniform thickness should not be limited to the absolute equality of the values. When the number of said concave-channels is numerous, the thickness of the side sealing-wall 143 can be 0.05˜0.25 mm thinner than the thickness of the inner sealing-wall 141 (or the outer sealing-wall 142) for convenience of measurement. The thickness value of the inner sealing-wall 141, the outer sealing-wall 142 and the side sealing-wall 143 is small, for convenience of quantification, the thickness ratio between the inner sealing-wall 141 (or the outer sealing-wall 142) and said side sealing-wall 143 within 1˜1.5, which still approximately consider that the thickness of the sealing wall 135 is substantially uniform and still does not deviate from the scope of the invention.

The sealing wall 135 in the present embodiment, comprises 8 linear normal concave-channels and 8 reverse concave-channels, however, a greater number or a smaller number of non-linear reverse concave-channel may be adopted. The side sealing-wall 143 of the present embodiment is substantially parallel to the longitudinal, axis 158, and in the lip-adjacent area, make a arbitrarily section plane that parallel to said axis 158 and meanwhile perpendicular to any one of said side sealing-walls 143, the intersected profile formed by said section plane and said concave-channels 140 and reverse concave-channels 150 is approximately U-shaped (the intersected profiles of other concave-channels are also defined in this way). That is, the section of the normal concave-channel 140 or the reverse concave-channel 150 is, approximately U-shaped. However, for convenience of manufacture, such as mold unloading, said side walls 143 may not be parallel to the longitudinal axis 158; that is, the section of said normal concave-channel 140 or the reverse concave-channel 150 is, approximately trapezoidal, even approximately triangle.

Taking the longitudinal axis 158 as a rotary axis, make a cylindrical surface with a radius R1 and intersects with said main rotary wall 138 to form an intersection, line, and create cutting plane M1 through said intersection line and perpendicular to the generating line of the main rotary wall 138 (with the axis 158 as rotary axis). Said cutting plane M1 divides the seal membrane 130 into an inner portion 156 (as in FIG. 11) and an outer portion 157 (FIG. 12). Said cutting plane M1 intersects the main rotary wall 138 to form a plurality of intersection lines 151a and 151b. Said cutting plane M1 intersects the side sealing-wall 143 to form a plurality of intersection lines 153a and 153b, and said cutting plane M1 intersects said outer sealing-wall 142 to form a plurality of intersection lines 152a and 152b. The plurality of segments 151a, 152a, 153a are formed an annular intersection line 155a; the plurality of segments 151b, 152b, 153b are formed an annular intersection line 155b, and the section 155 defined by said annular intersection line 155a and 155b.

As shown in FIG. 11-12, it is obvious that the circumference L1 of the intersection line 155a (155b) is much larger than 2*π*R1, that means the reverse concave-channel plays a role in enlarging hoop circumference, and the difference between L1 and 2*π*R1 is approximately equal to 2*P times the length L2 of the intersection line 153a (153b) (P is the number of reverse concave-channels). That is, the side sealing-wall 143 actually plays a role in enlarging hoop circumference. With the prerequisite of the reverse concave-channels width meeting the needs of the manufacturer, increasing the width of the reverse concave-channel does not mean have a larger hoop circumference. Those skilled in the art can understand that there must be some R1 value making the outer portion 157, which is divided by the cutting plane M1, to start from the section 155, the main change of its shape is shown as local bending deformation and macroscopic displacement of the seal membrane, rather than the overall microscopic molecular chain elongation and overall tensile deformation. And said inner portion 156, from said sealing lip 134 to said section 155, the change of shape is shown as the comprehensive effect of partial bending deformation and overall tensile deformation of the seal membrane. What it is quite clear is that said reverse concave-channels enlarge hoop circumference, and reduce the cylinder hoop strain (stress) when a large diameter instrument is inserted, thereby reducing the hoop force and the frictional resistance.

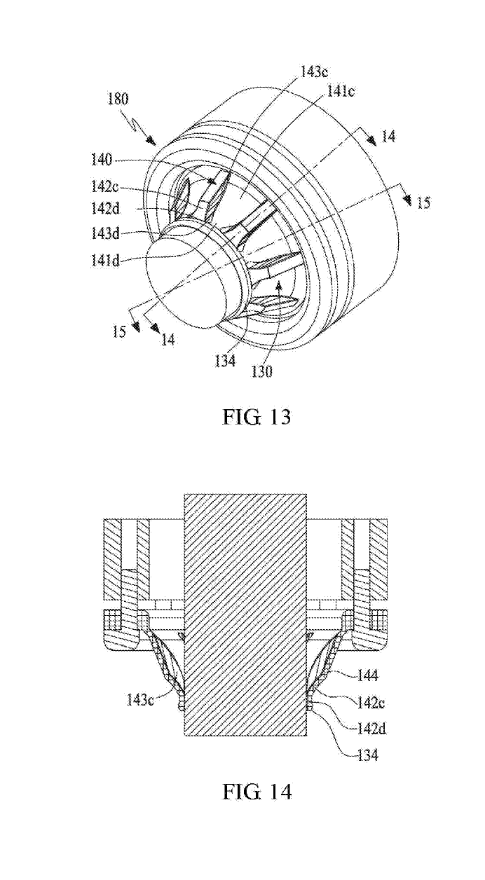

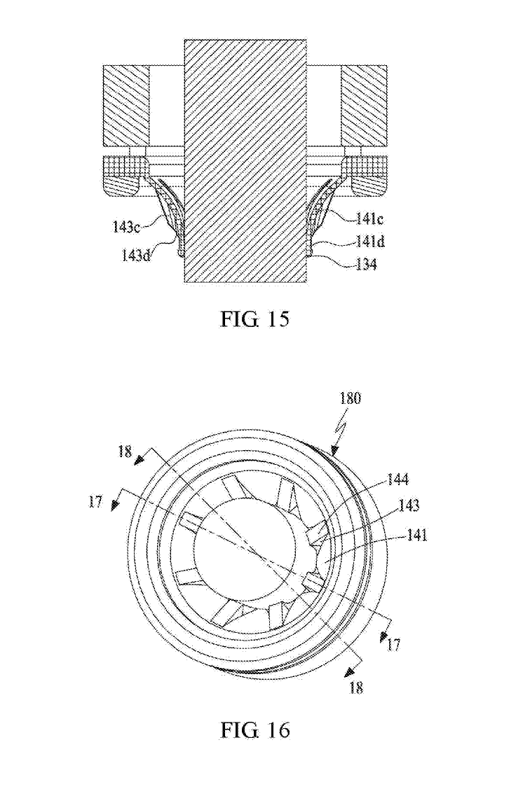

FIG. 13-15 shows a simulated deformation view of the seal membrane 130 when a large diameter instrument is inserted into said seal membrane assembly 180 (the floating portion outside the seal membrane and the protect device 160 are not shown). Said inner sealing-wall 141 is divided into two portions, an inner sealing-wall 141c and a cylindrical-sealing-wall 141d; said outer sealing-wall 142 is divided into two portions, an outer sealing-wall 142c and a cylindrical-sealing-wall 142d; said side sealing-wall 143 is divided into two portions, a side sealing-wall 143c and a cylindrical-sealing-wall 143d. Said cylindrical-sealing-wall 141d, said cylindrical-sealing-wall 142d, and said cylindrical-sealing-wall 143d together forms the wrapped, area around the outer surface of said inserted instrument. Studies have shown that, compared to the grooveless design, the wrapped area of the seal membrane with the concave-channel is small, and reducing the wrapped area can reduce the frictional resistance.

As FIG. 13-15 is shown when a large diameter instrument is inserted, only a small section of the concave-channel 140 is flattened. The unflattened concave-channel near the wrapped area has a better function of storing grease. When the instrument moves in the seal membrane, the grease in the wrapped area is scraped away firstly, and the grease in the unflattened concave-channel adjacent to the wrapped area will be added to the surface of the instrument, thereby adding to the wrapped area with the instrument moving. Optionally, the internal width of the concave-channel in, the lip-adjacent area is B1, wherein 0.5≤B1≤1 mm. When the inner width of the concave-channel in the lip-adjacent area is smaller than 0.5 mm, the structure of the concave-channel is hard to be manufactured; while the larger the internal width of the concave-channel, the worse the grease storage effect; Researches have shown that when the internal width of the groove is ≤1 mm, the grease storage effect is better. The grease storing in the concave-channel improves the problem of lubrication unreliability as described in the background, thereby contributing to reduce the stick-slip described in the background. The structure of said concave-channel enlarges hoop circumference, reduces the wrapped area when large instruments inserted, therefore, the frictional resistance can be reduced, to a large extent, so that the probability of the seal inversion occurrence described in the background can be reduced.

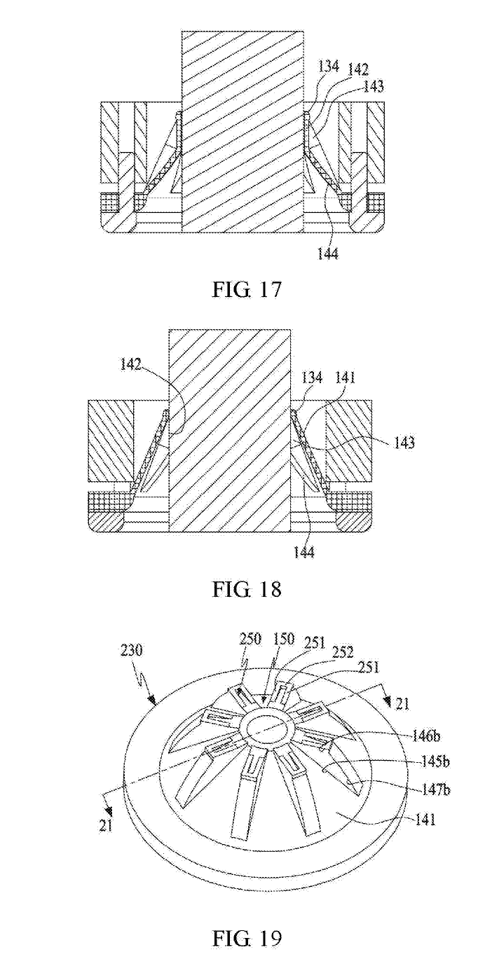

FIG. 16-18 shows a simulated deformation view of said seal membrane 130 (the floating portion outside the seal membrane and the protect device 160 are not shown) when said seal membrane assembly 180 is inverted. As mentioned above, the seal inversion is inevitable; after inversion happened, since the normal concave-channel 140 represented as a raised hollow convex-rib on the distal surface of the seal membrane, and in the lip-adjacent area, said reverse concave-channel 150 between, the ribs are formed, thereby reducing the actual contact area of the instrument and the inverted seal, membrane. Also, said inverted concave-channels enlarge hoop circumference, and reduce the cylinder hoop strain (stress) when a large diameter instrument is inserted, thereby reducing the hoop force and the frictional resistance. And thereby improving the operational comfort after the seal inversion to a large extent.

Referring to FIG. 7, FIG. 16-18, the depth of said normal concave-channel 140 gradually increases in the lip-adjacent area, and the depth of which rapidly decreases outside the lip-adjacent area, other advantages includes: simplify mould design, improve the efficiency of the seal membrane processing; reduce the space occupied by the lateral movement of the seal membrane assembly, so that the, size of trocar can be designed to be smaller; reduce the material accumulation between the seal membrane and the instrument after inversion and the actual contact area of the two surfaces.

Said side sealing-wall 143 has effects similar to reinforcing ribs those described in the background, all of the side walls 143 together reinforcing the axial tensile stiffness in the lip-adjacent area; and said side walls 143 increase the axial tensile stiffness without increasing the, hoop stiffness, thus increasing the axial stiffness without increasing the hoop force, such that which can effectively reduce the stick-slip described in the background. In this embodiment, 16 side walls 143 are included, while more or less side walls also can increase the axial tensile stiffness.

In summary, the structure of concave-channels has the functions of enlarging hoop circumference, reducing the wrapped area, reducing the actual contact area of the two surfaces between the instrument and the seal membrane, improving lubrication reliability, increasing the axial tensile stiffness, etc., thereby, the frictional resistance and the stick-slip can be greatly reduced, and the probability of inversion is reduced and the comfort of application is improved.

FIG. 19-21 show more detailed depiction the seal membrane 230 of the second embodiment in the invention. The numerical designations of the geometrical structure in FIG. 14-16 are the same as which in FIG. 8-10, it indicates that the structure of the same designations in the embodiment 2 and the embodiment I is basically equivalent. Said seal membrane 230 includes a distal aperture 133, a sealing lip 134, a sealing wall 135 and a flange 136, said distal aperture 133 formed by the sealing lip 134, said sealing wall 135 connecting the sealing lip 134 at one end and the flange 136 at the other end, said the seal membrane 130 including the proximal surface and the distal surface.

Defining the axis of said sealing lip 134 as the longitudinal axis 158, and a transverse plane 159 that is generally perpendicular to the longitudinal axis 158. Said wall 135 including an inner sealing-wall 141, an outer sealing-wall 142 and a side sealing-wall 143. Said inner sealing-wall 141 extends laterally from the sealing lip 134 to the flange 136; said an outer sealing-wall 142 extends laterally from the sealing lip 134 to tilted-sealing-wall 144; while said tilted-sealing-wall 144 and said inner sealing-wall 141 (or the flange 136) are intersected. The first side of said side sealing-wall 143 intersects the inner sealing-wall 141 and form a line 145a, 145b; the second side of said side sealing-wall 143 intersects the outer sealing-wall 142 to form a line 146a, 146b; the third side of said side sealing-wall 143 intersects the tilted-sealing-wall 144 to form a line of intersection 147a, 147b, One end of said tilted-sealing-wall 144 intersects the outer sealing-wall 142 to form a line 148a. 148b; The other end of said tilted-sealing-wall 144 intersects the inner side wall 142 to form a line 149a, 149b.

In the lip-adjacent area, said 2 adjacent side sealing-wall 143 and the outer sealing-wall 142 therebetween form a concave-channel that is recessed from the proximal surface toward the distal surface and the opening oriented to the proximal surface, which is defined as the normal concave-channel 140; meanwhile, said 2 adjacent side sealing-wall 143 and the inner sealing-wall 141 there between form a concave-channel that is recessed from the distal surface toward the proximal surface and the opening oriented to the distal surface, which is defined as the reverse concave-channel 150. Said inner sealing-wall 141, said side sealing-wall 143 and said outer sealing-wall 142 forms a series of normal concave-channel and reverse concave-channel, and said normal concave-channel and reverse concave-channel alternately distributed around the sealing lip 134, extending laterally outward and gradually increasing in axial depth. In the lip-adjacent area, a series of concave-channels with the normal and reverse alternately and with the increasing axial depth form a seamless sealing wall 135.

In the lip-adjacent area, the side sealing-wall 143 is a surface defined by both sides and extending laterally outward from the sealing lip 134 and gradually widening; while outside the lip-adjacent area, said side sealing-wall 143 is a tapered region defined by the sides, said side sealing-wall 143 is approximately obtuse triangular near the boundary of the lip-adjacent area. That is, when said normal concave-channel 140 extending laterally outward, the depth of it gradually increases in the lip-adjacent area, while gradually decreases outside the lip-adjacent area. Moreover, the 2 adjacent side walls 143 and the inner sealing-wall 141 there between only a distinct reverse concave-channel is formed in the lip-adjacent area, while no distinct reverse concave-channel is formed outside the lip-adjacent area. From the perspective of the distal surface, said normal concave-channel 140 is represented by a hollow convex-rib that is raised on the distal surface. Said rib extends laterally outward from the sealing lip and gradually increase in height, and abrupt change occurs near the boundary of the lip-adjacent area, and the height thereof rapidly decreases.

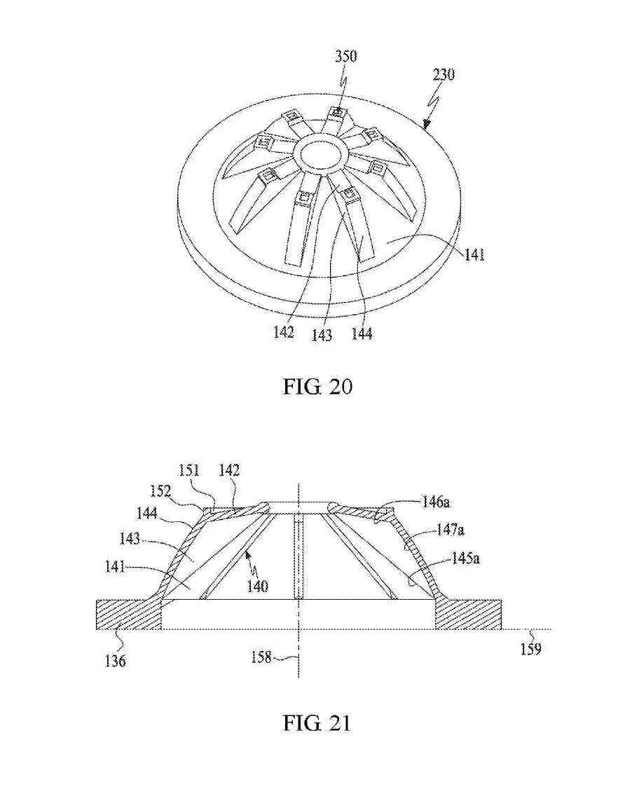

Optionally, the seal membrane 230 has a U-shaped solid-rib raised from the distal surface B of the outer sealing-wall 142. The U-shaped solid-rib is formed by two adjacent ribs 251 and a rib 252 there between. Although the sectional shape of the U-shaped ribs in the present embodiment approximates a U-shape, it may be approximately trapezoidal, other open polygons, circular rings or other closed polygons. FIG. 20 depicts a rectangular solid-rib raised from the, distal surface B of the outer sealing-wall 142. The main function of said U-shaped, rib or, rectangular, rib is: when inversion happened, the raised rib reduces surface direct contact between the instrument and the inverted seal membrane, thereby reducing the frictional resistance. At the same time, the recessed area between the ribs has a certain role in storing grease. When the seal membrane is turned over, the friction between the instrument and the seal membrane, first take away the grease on the raised ribs, while the grease in the recessed area between the ribs can be added to the surface of the rib as the instrument moves, thereby improve lubrication reliability in a certain degree after inversion. However, arbitrary rib cannot extend to the sealing lip 134, it should be kept away from the sealing'lip as much as possible to prevent from increasing the hoop force in adjacent areas.

Those skilled in the art easily understand that the reasonable fillet transition can avoid stress concentration, or make certain areas defaulted more easily. Due to the small size of the seal membrane, especially the area near the sealing lip is smaller, with such, a small size and different chamfer, the shape of the seal membrane looks different. In order to clearly show the geometric relationship of the elements, the embodiment of the invention is generally the pattern without the fillet.

Many different embodiments and examples of the invention have been shown and described. One of those ordinary skilled in the art will be able to make adaptations to the methods and apparatus by appropriate modifications without departing from the scope of the invention. The structure and the fixing manner of the protector assembly disclosed in U.S. Pat. No. 7,788,861 are used in the example of the present invention. While the structure and the fixing manner of the protector assembly disclosed in U.S. Pat. Nos. 5,342,315, 7,988,671, or US20050131349A1 can be used; or simply modify the fixing manner of the protector; in some applications, the protector assembly may not be included. The approximate U-shaped concave-channels and the approximate V-shaped concave-channels described in this embodiment cannot be limited to U-shaped or V-shaped. It has been mentioned many times in the invention that the concave-channel extends laterally outward from the sealing lip, and the so-called “extending laterally outward” should not be limited to a straight line. Said “extending laterally outward” can be a spiral, a line segment, a multi-section arc line and so on. In the invention, the positional relationship of the intersecting surfaces composed <of said concave-channel and the intersection line thereof are described with reference to specific embodiments, and the methods of increasing curved surfaces to form a multifaceted mosaic or using of the high-order curved surface to make the intersection line and the concave-channel shape to look different from said embodiment. However, it can be considered not deviated from the scope of the invention, as long as it conforms to the general idea of the invention. Several modifications have been mentioned, to those skilled in the art, other modifications are also conceivable. Therefore, the scope of the invention should follow the additional claims, and at the same time, it should not be understood that it is limited by the specification of the structure, material or behavior illustrated and documented in the description and drawings.

I claim:

1. A trocar sealing system comprising a seal membrane, the seal membrane comprising a proximal opening, a distal aperture, and a sealing wall which extends from the distal aperture to the proximal opening, the the sealing wall comprising a proximal surface and a distal surface, the distal aperture formed by a sealing lip for accommodating the inserted instrument and forming a gas-tight seal; wherein
the sealing wall comprising a plurality of normal concave-channels and a plurality of reverse concave-channels alternately distributed around the sealing lip;
the normal concave-channel is recessed from the proximal surface of the sealing wall toward the distal surface and the opening oriented to the proximal surface; The shape of the normal concave-channel from the perspective of distal surface is represented as a hollow convex-rib that is raised from the distal surface;
the normal concave-channels extending laterally outward from the sealing lip, and the depth of concave-channels gradually increase in the lip-adjacent area; while the depth of concave-channels gradually decrease outside the lip-adjacent area.

2. The trocar sealing system according to claim 1, wherein the section of the normal concave-channel is U-shaped.

3. The trocar sealing system according to claim 1, comprising eight normal concave-channels.

4. The trocar sealing system according to claim 1, the seal membrane includes a flange at which the sealing wall extendedly intersects, or simultaneously the sealing wall and the concave-channel extendedly intersect, and an outer floating portion including at least one lateral pleat extending from the flange to the proximal opening.

5. The trocar sealing system, according to claim 4, comprising:
a protection device, the first retainer ring and the second retainer ring; The protection device comprises a retainer flange, the seal membrane and the protect device are sandwiched between the first retainer ring and the second retainer ring,
and one side of the flange of the protection device is in close contact with the flange of the seal membrane, while the other side in close contact with the first retainer ring; the geometric size of the first retainer ring conforms to the following equation:
Rᵢ≥Rᵢₙ+Tₛ+Tₚ+δ
H≥Hₛ+ΔL
wherein:
Ri=radius of center hole of the first retainer ring;
Rin=the largest radius designed for the surgical instrument passed through the seal membrane;
Ts=thickness of sealing wall;
Tp=thickness of protector;
δ=reserved clearance;
H=height of the first retainer ring;
Hs=vertical height of a frustum section of the seal membrane;
ΔL=elongation of a frustum section of the seal membrane when working.

6. The trocar sealing system according to claim 1, in the lip-adjacent area, the sealing wall comprising a plurality of inner sealing-walls, side sealing-walls and outer sealing-walls; two side sealing-walls and one outer sealing-wall from a normal concave-channel; two side sealing-walls and one inner sealing-wall form a reverse concave-channel.

7. The trocar sealing system according to claim 6, the adjacent normal concave-channel and the reverse concave-channel sharing a mutual side sealing-wall.

8. The trocar sealing system according to claim 6, the thickness of the inner sealing-walls, side sealing-walls and outer sealing-walls are substantially uniform.

9. The trocar sealing system according to claim 8, in the lip-adjacent area, the side sealing-wall is a surface defined by both edges and extending laterally outward from the sealing lip and gradually widening; while outside the lip-adjacent area, the side sealing-wall is a tapered region defined by the edges; the side sealing-wall is approximately obtuse triangular near the boundary of the lip-adjacent area.

10. The trocar sealing system according to claim 9, when a large diameter surgical instruments inserting into the trocar, the side sealing-walls function as enlarging hoop circumference and increasing the axial tensile stiffness; and reducing the actual contact area of the two surfaces between the instrument and the seal membrane; and reducing the wrapped area.

11. The trocar sealing system of claim 6, the internal width of the normal concave-channel in the lip-adjacent area is B and 0.5≤B≤1 millimeter.

12. The trocar sealing system according to claim 11, wherein the sealing system also include lubricating grease, and a function of the normal concave-channel is storing the grease; when a large diameter surgical instruments inserting into the trocar, a small section of the normal concave-channel is flattened and the other section of the normal concave-channel unflattened; the grease storing in the normal concave-channel benefit of adding to the flattened section from the unflattened section so as to improve lubrication reliability.

13. A trocar sealing system according to claim 1, wherein in the lip-adjacent area, the normal concave-channel includes two side sealing-walls and an outer sealing-wall defined by the two side sealing-walls, the outer sealing-wall has a proximal surface and a distal surface, and the outer sealing-wall includes solid-rib rising from the distal surface.

14. The trocar sealing system according to claim 13, wherein the solid-rib formed by two adjacent ribs and a rib there between and approximately U-shaped.

15. The trocar sealing system according to claim 13, wherein the solid-rib does not extend to the sealing lip, the solid-ribs function as reducing surface direct contact between the instrument and the inverted seal membrane when sealing system inversion happened.

16. The trocar sealing system according to claim 13, four solid-ribs raised from the distal surface of the outer sealing-wall and form a rectangular solid-rib.

17. The trocar sealing system according to claim 16, wherein the rectangular solid-ribs function as reducing surface direct contact between the instrument and the inverted seal membrane when sealing system inversion happened; and the recessed area between the ribs has a certain role in storing grease.
